(19) United States
(12) Patent Application Publication
(10) Pub. No.: US 2007/0088291 A1
(43) Pub. Date: Apr. 19, 2007

(54) Syringe construction

(76) Inventor: Eugene E. Weilbacher, Chesterfield, MO (US)

Correspondence Address: CARTER, DELUCA, FARRELL & SCHMIDT, LLP, 445 BROAD HOLLOW ROAD, SUITE 225, MELVILLE, NY, 11704, US

(21) Appl. No.: 11/517,043

(22) Filed: Sep. 7, 2006

Related U.S. Application Data
(60) Provisional application No. 60/714,911, filed on Sep. 7, 2005.

(51) Int. Cl. A61M 5/315

(52) U.S. Cl. 604/218

(57) ABSTRACT

A syringe assembly is provided comprising an annular syringe body, a plunger adapted to travel therein in response to manual force, said plunger terminating in an elastomeric seal in sealing engagement with the interior surface of the syringe body, said seal having admixed therein a lubricant capable of migrating through the elastomeric seal to bloom on the surface thereof, thereby imparting enhanced lubricity thereto reducing the movement forces necessary to enable controlled plunger movement.

This application claims priority from U.S. Provisional Application Ser. No. 60/714,911 filed Sep. 7, 2005, the contents of which is incorporated herein by reference in its entirety.

### BACKGROUND

1. Technical Field

This invention relates to an improved syringe construction; more particularly, this invention relates to a syringe construction exhibiting reduced plunger movement forces.

2. Background of the Related Art

Syringes are typically constructed of an annular syringe body, a plunger adapted to travel within the syringe body in response to manually applied force, and an annular needle removably attached to the distal portion of the syringe body. The plunger is basically a piston terminating in an elastomeric seal. The syringe can either be pre-filled or can be filled by inserting the needle into a vial of the liquid to be drawn into the syringe and withdrawing the plunger to thereby draw the liquid into the syringe body in the region between the elastomeric seal and the needle.

Plungers in syringes are know to exhibit greater than desired movement forces especially when the syringes have been pre-filled and stored for a period of time. The movement forces include what is known as the breaking force, i.e., the relatively high force needed to move the plunger after is has been positioned in one location in the syringe body for an extended period of time. The movement forces also include the extrusion of running force, i.e., the force required to move the plunger within the syringe body at a desired rate. Since syringes are used primarily to administer drugs in specified doses, reduced plunger movement forces are desirable to provide better control of the dose delivered. Reduction in movement forces is especially desired in syringes that are pre-filled with a liquid.

In the past, efforts have been made to reduce movement forces primarily by applying lubricant coatings to the interior surface of the syringe body and the surface of the elastomeric seal of the plunger which contacts the syringe body. For example, most medical syringes employ a coating of silicone oil on the interior of the syringe body and the seal of the plunger. While the silicone coating reduces plunger movement forces, it is not acceptable for all applications because of potential contamination of the medicinal liquid within the syringe. Further efforts have been made to reduce plunger movement forces by coating the interior surface of the syringe body with a polymeric coating such as with a coating of a para-xylylene polymer (“parylene”) (see, U.S. patent application Publication No. US 2005/0010175 A1 published Jan. 13, 2005). Applying parylene to the elastomeric seal and/or the interior surface of the syringe body significantly reduces plunger movement forces as compared to simply coating the seal and/or interior surface of the syringe body with silicone oil. The parylene coating, however, is applied by vapor deposition which is not only an additional process step but also presents a significant added expense.

### SUMMARY

Accordingly, it is an object of the present invention to provide an improved syringe construction which enables reduced plunger movement forces to be obtained in a simple, low cost manner.

This, as well as other objects and advantages are accomplished by the present invention which provides a syringe assembly comprising an annular syringe body and a plunger adapted to travel therein, said plunger terminating in an elastomeric seal, said seal having admixed therein during fabrication of the seal, a lubricant capable of migrating through the elastomeric seal to bloom on the surface thereof thereby imparting enhanced lubricity thereto reducing the movement forces necessary to enable controlled plunger movement.

### BRIEF DESCRIPTION OF THE DRAWINGS

The present invention is illustrated in the accompanying drawings, wherein:

FIG. 1 is a cross sectional elevation view of one embodiment of a syringe of the present invention; and

FIG. 2 is an enlarged cross sectional partial elevation view of the syringe of FIG. 1 illustrating the migration and blooming of the lubricant in the elastomeric seal;

### DETAILED DESCRIPTION OF THE INVENTION

While this invention is susceptible of many different embodiments, certain preferred embodiments will be described herein in detail with the understanding that the present disclosure is to be considered as providing various exemplifications of the principles of the present invention and is not intended to limit the invention to be specific embodiments illustrated herein.

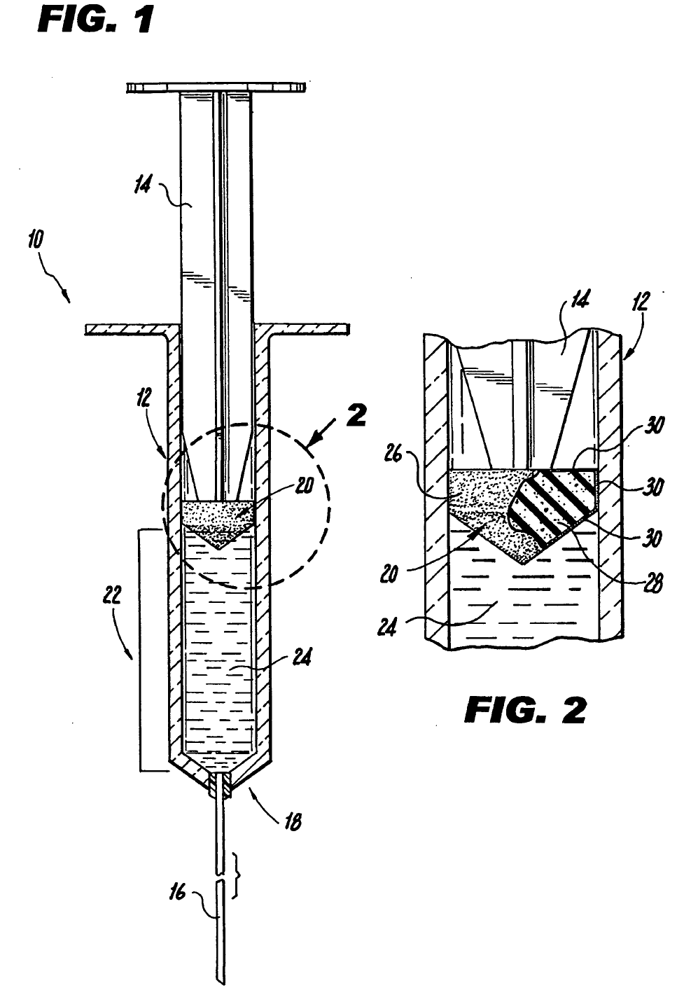

Referring now to FIG. 1, a syringe 10 of the present invention is shown as comprising an annular syringe body 12, a plunger 14 adapted to travel within the syringe body 12 in response to manually applied force, and an annular needle 16 removably attached to the distal portion 18 of the syringe body 12. The plunger 14 is basically a piston terminating in an elastomeric seal 20. The region 22 bounded by the syringe body 12, the distal portion 18 of the syringe body 12 and the distal portion of the elastomeric seal 20 can either be pre-filled with a liquid 24 which is typically a medicinal solution or can be filled with such liquid 24 by inserting the needle 16 into a vial containing the liquid 24 (not shown) and withdrawing the plunger 14 to thereby draw the liquid 24 into region 22.

Syringe bodies are typically manufactured from glass or polymeric resins. Typical polymeric resins used in the manufacture of syringe bodies include olefin polymers and copolymers, polystyrene, polycarbonate, acrylate or methacrylate copolymers, cyclic olefin-containing polymers, bridged polycyclic hydrocarbon containing polymers, (see, for example, U.S. Pat. No. 6,085,270), and the like. Similarly, the plunger also can be manufactured from glass or polymeric resins. The plunger terminates in an elastomeric seal which deforms in use to provide a seal against the inner surface of the syringe body. The elastomeric seal can be affixed to the distal end of the plunger in any known manner, e.g., it can be adhesively secured thereto, threadably engaged thereto, frictionally engaged thereto, and the like.

The elastomeric seal can be formed from any elastomeric material which is generally inert and impervious with regard to the medicinal fluids anticipated to be used in conjunction with the syringes of the present invention. Suitable elastomers include natural rubber, styrene-butadiene rubber, acrylonitrile-butadine copolymers, neoprene, butyl rubber, polysulfide elastomers, urethane rubbers, ethylene-propylene diene (EPDM) elastomers, and the like.

In the fabrication of the elastomeric seal, the elastomeric material can be compounded with a variety of additives in, for example a Banbury mixer, to incorporate therein antioxidants, UV stabilizers, colorants, metallic stearates such as zinc, calcium, magnesium, lead and lithium stearates, fluoropolymers such as powered poly (tetrafluoroethylene) (TEFLON), and the like.

It has been found that when fluoropolymer additives are employed, unlike the lubricants useful in the present invention, the fluoropolymers do not migrate to the surface of the elastomeric material. Instead, the fluoropolymer additives become part of the matrix of the molded elastomeric material. The fluoroploymer additives are compounded with the elastomeric material in amounts ranging from about 10 to 50% by weight and preferably, from about 20 to 30% by weight based on the weight of elastomer. Incorporation of the fluoropolymer additives has been found to result in an elastomeric material exhibiting a reduced coefficient of friction.

In accordance with the present invention, during the compounding operation, one or more lubricants can be admixed with the elastomeric material to incorporate the lubricant(s) therein. Suitable lubricants are those that are not compatible with the elastomer and will migrate through the elastomer and bloom on the surface thereof. Continued blooming of the lubricant will provide the necessary lubricity to reduce the coefficient of friction of the seal and thereby reduce plunger movement forces providing an improved syringe construction. Lubricants useful in the present invention include, for example, both organic and inorganic lubricants. Exemplary organic lubricants include amides, especially oleamides, waxes, both natural and synthetic, for example, bees wax and derivatives of hydrogenated castor oil such as methyl 12-hydroxystearate, esters, oils such as mineral oils and the like. Inorganic lubricants include, for example graphite in flake or powered form, molybdenum disulfide, and the like.

The lubricant can be admixed with the elastomeric material in amounts ranging from about 0.05 to 0.50 wt % based on the weight of the elastomeric material and preferably, from about 0.2 to 0.4 wt %.

As shown in FIG. 2, the plunger 14 terminates in elastomeric seal 20. The elastomeric seal comprises elastomeric material 26 having a lubricant 28 admixed therein. The lubricant 28 migrates through the elastomeric material and blooms on the surface thereof forming a lubricious coating 30. After compounding, the resulting elastomer can be thermoformed to the desired size and shape and affixed to the distal end of the plunger 14. The resulting plunger assembly can be inserted into syringe body 12 to form the improved syringe construction of the present invention.

Syringes fabricated in accordance with the present invention have been found to exhibit a plunger breaking force reduction of about 75% and a running force reduction ranging between about 10 and 20%.

It will be understood that the invention may be embodied in other specific forms without departing from the spirit of scope thereof. The presently disclosed embodiments, therefore, are to be considered in all respects as illustrative and not restrictive, and the invention is not to be limited to the details set forth herein.

What is claimed is:

1. A syringe assembly comprising an annular syringe body, a plunger adapted to travel therein in response to manual force, said plunger terminating in an elastomeric seal in sealing engagement with the interior surface of the syringe body, said seal having admixed therein a lubricant capable of migrating through the elastomeric seal to bloom on the surface thereof, thereby imparting enhanced lubricity thereto reducing the movement forces necessary to enable controlled plunger movement.

2. A syringe assembly as defined in claim 1, wherein the syringe body and plunger are fabricated from glass.

3. A syringe assembly as defined in claim 1, wherein the syringe body and plunger are fabricated from a polymeric resin.

4. A syringe assembly as defined in claim 1, wherein the elastomeric seal is fabricated from an elastomer selected from the group consisting of natural rubber, sytpene-butadiene rubber, acrylonitrile-butadine copolymer, neoprene, butyl rubber, polysulfide elastomer, urethane rubber, ethylene-propylene diene elastomer, and mixtures thereof.

5. A syringe assembly as defined in claim 1, wherein the lubricant is an organic lubricant selected from the group consisting of amides, waxes, esters, oils and mixtures thereof.

6. A syringe assembly as defined in claim 5, wherein the lubricant is admixed with the elastomeric material in amounts ranging from about 0.05 to 0.50 wt % based on the weight of the elastomeric material.

7. A syringe assembly as defined in claim 6, wherein the lubricant is admixed with the elastomeric material in amounts ranging from about 0.2 to 0.4 wt % based on the weight of the elastomeric material.

8. A syringe assembly as defined in claim 1, wherein the elastomer additionally containing one or more additives selected from the group consisting of antioxiants, UV stabilizers, colorants, metallic stearates, fluoropolymers, and mixtures thereof.

9. A syringe assembly as defined in claim 8, wherein the fluoropolmer is present in amounts ranging from about 10 to 50% by weight based on the weight of elastomer.

10. A syringe assembly as defined in claim 9, wherein the fluoropolymer is present in amounts ranging from about 20 to 30% by weight based on the weight of elastomer.
